(19) United States
(12) Patent Application Publication
(10) Pub. No.: US 2020/0017482 A1
(43) Pub. Date: Jan. 16, 2020

(54) SURFACTANT SYSTEMS FOR CRYSTALLIZATION OF ORGANIC COMPOUNDS

(71) Applicant: Novartis AG, Basel (CH)

(72) Inventors: Fabrice GALLOU, Basel (CH); Michael PARMENTIER, Basel (CH)

(21) Appl. No.: 16/334,970

(22) PCT Filed: Sep. 26, 2017
(86) PCT No.: PCT/IB2017/055837

§ 371 Date: Mar. 20, 2019

Related U.S. Application Data
(60) Provisional application No. 62/400,311, filed on Sep. 27, 2016.

(51) Int. Cl. C07D 405/14; C07B 63/04

(52) U.S. Cl. CPC C07D 405/14; C07B 2200/13; C07B 63/04

(57) ABSTRACT

The present invention is directed to methods for producing crystals or aggregates of organic compounds using a composition comprising a surfactant-water mixture, the organic compound and optionally an organic solvent as starting composition. This surfactant system enables the production of a high variety of different crystals and aggregates. The method is in particular suitable for obtaining crystals and aggregates optimized for isolation and purification during chemical synthesis processes.

### FIELD OF THE INVENTION

The present invention is directed to methods for crystallizing or aggregating organic compounds. Organic compounds are crystallized or aggregated using a composition comprising a surfactant-water mixture. This method can in particular be used in synthesis and purification of said organic compounds.

### BACKGROUND OF THE INVENTION

Precipitating/crystallizing organic compounds from solution is a common and well known means for isolation and purification of said organic compounds, for example during chemical synthesis processes are for their purification. However, in many cases the obtained crystals or aggregates are unfavorable for processing, leading to long filtration times, or handling problems, or display sub-optimal formulating properties such as wettability for example. Changing of the form and size of the crystals and aggregates of organic compounds therefore is it tool to optimize and in particular accelerate the production of organic compounds. The possibilities to alter the properties of the crystals or aggregates, however, are often limited by the technology available for their production. Therefore, there is a need in the art to provide new methods for producing crystals and aggregates of organic compounds which offer the possibility to easily change their properties.

### SUMMARY OF THE INVENTION

The present invention is based on the findings that crystallization and aggregation of organic compounds out of mixtures comprising a surfactant results in different specific types of crystals/aggregates depending on the used surfactant. Some physical properties such as the shape, size, and wetting of the crystals/aggregates can further be controlled by the addition of co-solvents.

In a first aspect, the present invention provides a method of producing crystals or aggregates of organic compounds, comprising providing a composition comprising an organic compound and a surfactant-water mixture, and inducing crystallization or aggregation of the organic compound.

In a second aspect, the present invention provides a method for synthesizing an organic compound, comprising chemically modifying a starting compound to produce said organic compound, and isolating the obtained organic compound using the method for producing crystals or aggregates according to the first aspect.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### DETAILED DESCRIPTION OF THE INVENTION

The present inventors demonstrated that crystallization/aggregation of organic compounds from aqueous mixtures comprising a surfactant leads to the formation of different crystal and aggregate forms of the organic compound, depending on the surfactant used, its concentration, and the type and concentration of a co-solvent which may optionally be used. Thereby, crystal/aggregate forms of the organic compound are available which can easily and very rapidly be isolated. For example, an organic compound which was only obtainable in one crystallized form by conventional means needed very long filtration times during purification (up to 10 minutes at laboratory scale). Using the methods according to the present invention, filtration time could be reduced to 5 seconds (at laboratory scale) due to optimized crystal/aggregate forms.

The present invention provides, in a first aspect, a method of producing crystals or aggregates of an organic compound, comprising providing a composition comprising said organic compound and a surfactant-water mixture, and inducing crystallization or aggregation of the organic compound.

The inventive technology is generally applicable for any organic compound which is at least partially water-soluble. The described methods in particular are suitable for organic compounds of smaller size (small molecules) which may have a molecular weight of up to 2,000 Dalton, especially up to 1,500 Dalton or even only up to 1,000 Dalton. In certain embodiments, the organic compound is not a protein, peptide or nucleic acid. For example, the organic compound may be an intermediate or final product of a chemical synthesis process.

In specific embodiments, the organic compound is only partly water-miscible. An organic compound which is only partly water-miscible in particular is only miscible with water at a concentration of 20 WI or less, especially 10 WI or less or 5 WI or less, at room temperature. The organic compound can be present in the aqueous mixture in any concentration which is feasible for performing the method. In particular, the organic compound is used at high concentrations. For example, the concentration of organic compound in the aqueous mixture is at least 0.1 M, in particular at least 0.5 M, at least 1.0 M, at least 1.1 M, at least 1.2 M, at least 1.3 M, at least 1.5 M, at least 1.7 M or at least 2.0 M.

The organic compound is provided in a composition comprising a surfactant-water mixture. In particular, this composition may be obtained by solving the organic compound in a surfactant-water mixture. Alternatively, chemical synthesis of the organic compound may be performed in a surfactant-water mixture so that the product of the synthesis reaction is a respective composition comprising the organic compound and the surfactant-water mixture. After or together with contacting the organic compound with the surfactant-water mixture, an organic solvent may be added to the composition. In another embodiment, the organic compound is first solved in an organic solvent or synthesized in an organic solvent, and then a surfactant-water mixture is added to the composition. The organic solvent is discussed in detail below.

In certain embodiments, crystallization or aggregation of the organic compound is induced in the method by reducing the solubility of the organic compound in the composition. Crystallization or aggregation of the organic compound may be induced by various means or combinations thereof, including by reducing the temperature of the mixture, by increasing the concentration of the organic compound in the composition, and by adding an anti-solvent. For example, in specific embodiments the temperature of the composition is decreased by at least 10° C., especially at least 15° C., at least 20° C., at least 25° C. or at least 30° C. for inducing crystallization or aggregation, for example from about 50 or 40° C. to about 20° C. In further embodiments, the concentration of the organic compound in the composition is increased for inducing crystallization or aggregation. This may be achieved by adding further organic compound or by removing other components from the composition, in particular by removing water, e.g. through evaporation. In further embodiments, an anti-solving agent is added to the composition for inducing crystallization or aggregation. The anti-solvent reduces the solubility in of the organic solvent in the composition. Suitable anti-solvents include, for example, water, or an additive already present in the composition.

The surfactant in the surfactant-water mixture can be any surfactant. In certain embodiments, the surfactant is a non-ionic surfactant. The surfactant generally is amphiphilic and comprises a hydrophilic part and a hydrophobic part. In specific embodiments, the surfactant is able to form micelles in the surfactant-water mixture.

In certain embodiments, the hydrophilic part of the surfactant comprises a polyalkylene glycol moiety, especially a polyethylene glycol moiety or a polypropylene glycol moiety. The polyalkylene moiety, especially the polyethylene glycol moiety, may have an average molecular weight in the range of about 100 to about 10,000 g/mol, especially in the range of about 300 to about 3,000 g/mol, in particular in the range of about 400 to about 2,000 g/mol. Certain examples of surfactants comprising a polyalkylene glycol moiety include tocopherol polyethylene glycol succinates (TPGS), in particular DL-α-tocopherol polyethylene glycol succinates such as TPGS-750-M, TPGS-1000, TPGS-1500, TPGS-400, TPGS-1100-M, TPGS-2000, TPGS-860-oleate, TPGS-PEG-PPG-PEG-1100 and TPGS-PPG-PEG-70-butyl, and DL-α-tocopherol polypropylene glycol succinates such as TPPG-1000 and TPPG-1000-butyl; Triton X-100; polyethylene glycol alkyl ethers such as Brij surfactants, in particular Brij 30, Brij 35, Brij 52, Brij 56, Brij 58, Brij 72, Brij 76, Brij 78, Brij 92, Brij 96, Brij 98, Cremophor A6, Cremophor A25 and Thesit; polyethylene glycol esters such as polyethylene glycol (15)-hydroxystearate (Solutol HS 15); polyethylene glycol sorbitan fatty acid esters, also known as polysorbates or Tween, such as polysorbate 20, polysorbate 21, polysorbate 40, polysorbate 60, polysorbate 61, polysorbate 65, polysorbate 80, polysorbate 81, polysorbate 85 and polysorbate 120; cholesteryl PEG succinates such as holesteryl PEG1000 succinate; (deoxy) cholic PEG such as colic PEG1000 and deoxy-cholic PEG1000; chromanol polyethylene glycol succinates such as Chrom-400 and Chrom-1000; b-sitosterol methoxyethyleneglycol succinate (Nok); other derivatives of PEG such as C4-azo-PEG; polyethylene glycol such as PEG200, PEG600 and PEG1000 (PEG with an average molecular weight of 200 g/mol, 600 g/mol and 1000 g/mol, respectively); and polypropylene glycole. In specific embodiments, the surfactant is a DL-α-tocopherol polyethylene glycol succinate, in particular TPGS-750-M.

Furthermore, also other surfactants can be used, including, for example, cetyltrimethylammonium bromide (CTAB); phase transfer surfactants (PTS) such as sodium deoxycholate; polyoxyethanyl ubiquinol sebacate (PQS) and functionalized PQS; and octanoic acid and other long alkyl chain acids, in particular C6-C20 alkyl chain acids.

The concentration of the surfactant in the surfactant-water mixture in particular is in the range of 0.1 to 10% (w/w). In certain embodiments, the concentration of the surfactant in the surfactant-water mixture is in the range of 0.5 to 5% (w/w), especially in the range of 0.8 to 4% (w/w), 1 to 3% (w/w) or 1.5 to 2.5% (w/w), such as about 2% (w/w). In specific embodiments, the concentration of the surfactant in the surfactant-water mixture is above its critical micellar concentration.

In certain embodiments, the composition further comprises an organic solvent. The organic solvent in the composition may be any organic solvent. In certain embodiments, the organic solvent is water-miscible or partly water-miscible. Suitable examples of the organic solvent include alcohol such as C₁₋₁₂ aliphatic alcohols, in particular C₁₋₈ aliphatic alcohols or C₁₋₈ aliphatic alcohols or C₁₋₄ alcohols, especially methanol, ethanol, n-propanol, and isopropanol; acetone; tetrahydrofuran (THF) and derivatives thereof such as methyl tetrahydrofuran; pyridine; acetonitrile; dimethylsulfoxide (DMSO), dimethylformamide (DMF); dichloromethane (DCM); and toluene.

The concentration of the organic solvent in the composition in particular may be in the range of 0.2 to 90% (w/w). In certain embodiments, the concentration of the organic solvent in the composition is in the range of 0.5 to 80% (w/w), especially in the range of 1 to 75% (w/w). In some embodiments, in particular where the organic solvent is added to the organic compound together with or after the surfactant-water mixture, the concentration of the organic solvent in the composition is in the range of 0.5 to 25% (w/w), especially in the range of 1 to 20% (w/w), 1.5 to 15% (w/w) or 2 to 12.5% (w/w), such as about 2% (w/w), about 5% (w/w) or about 10% (w/w). In other embodiments, in particular where the organic compound is first contacted with, in particular solved in the organic solvent before the surfactant-water mixture is added, the concentration of the organic solvent in the composition is in the range of 20 to 90% (w/w), especially in the range of 25 to 80% (w/w), 30 to 75% (w/w) or 40 to 70% (w/w), such as about 50% (w/w), or about 66% (w/w). In other embodiments, the above percentages are volume percentages (% (v/v)) instead of weight percentages (% (w/w)).

The method of producing crystals or aggregates of an organic compound may further comprise the step of obtaining the crystallized or aggregated organic compound from the composition. In particular, the crystal or aggregate is separated or isolated from the remaining parts of the composition. In certain embodiments, the crystallized or aggregated organic compound is obtained by filtration. The crystals or aggregates of the organic compound are held back by the filter while the other parts of the composition pass through the filter. More sophisticated means of filtration can be engineered.

The present technology may especially be used in industrial scale. The aqueous mixture may for example have a volume of at least 1 l, in particular at least 10 l, at least 100 l, or at least 1000 l.

The method for producing crystals or aggregates of organic compounds as described herein may also be used to recrystallize an organic compound. Hence, in another aspect the present invention provides a method for changing the morphology of crystals or aggregates of an organic compound, comprising solving the crystals or aggregates of the organic compound in a composition comprising a surfactant-water mixture, and inducing crystallization or aggregation of the organic compound.

Crystallization or aggregation of the organic compound may be performed using the method according to the first aspect of the present invention. All embodiments, examples and features described herein, including combinations thereof, for the method of producing crystals or aggregates of organic compounds also apply to the method for changing the morphology of crystals or aggregates of an organic compound. In particular, in certain embodiments the composition comprising a surfactant-water mixture further comprises an organic solvent.

In certain embodiments, the crystals or aggregates of the organic compound which morphology shall be changed were obtained at different conditions than those used in the method for changing the morphology of crystals or aggregates of an organic compound. In particular, these crystals or aggregates were obtained by crystallization or aggregation out of another composition, in particular out of a composition which does not comprise a surfactant-water mixture and an organic solvent, especially a composition which does not comprise a surfactant-water mixture.

The step of solving the crystals or aggregates of the organic compound in a composition comprising a surfactant-water mixture includes embodiments wherein the crystals or aggregates of the organic compound are first solved in an organic solvent and then a surfactant-water mixture is added to the solution, thereby forming said composition.

In a further aspect, the present invention provides a method for synthesizing an organic compound, comprising chemically modifying a starting compound to produce said organic compound, and isolating and/or purifying the obtained organic compound via crystallization or aggregation by providing a composition comprising a surfactant-water mixture and said organic compound and inducing crystallization or aggregation of the organic compound.

Isolation and/or purification of the organic compound may be performed using the method according to the first aspect of the present invention. All embodiments, examples and features described herein, including combinations thereof, for the method of producing crystals or aggregates of organic compounds also apply to the method for synthesizing an organic compound. In particular, in certain embodiments the composition comprising a surfactant-water mixture further comprises an organic solvent.

The step of providing a composition comprising a surfactant-water mixture and said organic compound may for example be performed by solving the organic compound in a surfactant-water mixture, or the organic compound may already be present in a surfactant-water mixture at the end of the chemical modification. In another embodiment, the organic compound may be solved in or already present at the end of the chemical modification in an organic solvent, and then a surfactant-water mixture may be added.

The chemical modification of the starting compound includes any chemical reaction suitable for producing the organic compound of interest. The organic compound may be an intermediate product or a final product of a chemical synthesis.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements may and/or indeed does encompass further elements as well as embodiments wherein the subject-matter which “comprises” specifically listed elements does not comprise further elements. Likewise, the expression “have” is to be understood as the expression “comprise”, also including and specifically referring to the expressions “consist essentially of” and “consist of”.

Numeric ranges described herein are inclusive of the numbers defining the range. The headings provided herein are not limitations of the various aspects or embodiments of this invention which can be read by reference to the specification as a whole. According to one embodiment, subject matter described herein as comprising certain steps in the case of methods or as comprising certain ingredients in the case of compositions refers to subject matter consisting of the respective steps or ingredients. It is preferred to select and combine specific aspects and embodiments described herein and the specific subject-matter arising from a respective combination of specific embodiments also belongs to the present disclosure.

### EXAMPLES

### Example 1: Conventional Protocol for Crystallization of Compound 1

500 mg (N-(4-(chlorodifluoromethoxy)phenyl)-6-((R)-3-hydroxypyrrolidin-1-yl)-5-(1-(tetrahydro-2H-pyran-2-yl)-1H-pyrazol-5-yl)nicotinamide) obtained from liquid-solid chromatography (LSC) was solved in MeOH (16v) at 60° C. under mechanical stearing to obtain a clear solution. Anti-solvent (8v) was added and after 20 min stirring a cooling ramp was initiated by switching off the oil bath (˜500 mL) to RT (˜22° C.). The reslurry was carried out in anti-solvent (8v) at 40° C. for 24 h. The obtained crystals were filtered on a frit (Por. 4) with vacuum.

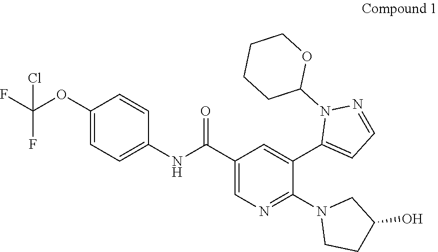 [SMILES: O=C(Nc1ccc(OC(F)(F)Cl)cc1)c1cnc(N2CC[C@@H](O)C2)c(-c2ccnn2C2CCCCO2)c1]

The conventional protocol for crystallization of compound 1 led to very long filtration times and poor control of distribution.

### Example 2: Crystallization of Compound 1 with TPGS-750-M

Starting material (compound 1): Columnar particles from ˜1 μm up to 15 μm in length. Surface overall smooth edges, mostly regular. Particles often lengthwise aggregated. Most of the particles are smaller than 8 μm. XRPD: Form A

500 mg of compound 1 was added to solution of TPGS-750-M in water (2% wt) (16 v) at 23° C. The resulting suspension was warmed to 40° C. and stirred for 24 h. The mixture was cooled to 23° C., stirred and the suspension was filtered under vacuum (Por. 4).

Obtained material: Aggregates/agglomerates (up to 300 μm) of columnar particles from ˜1 up to 10 μm in length. Surface both smooth and rough, edges irregular. Most of the particles are smaller than 6 μm. XRPD: Form A. Filtration time on lab scale: >5 min

### Example 3: Crystallization of Compound 1 with TPGS-750-M and 10% Wt MeOH

500 mg of compound 1 was added to solution of TPGS-750-M in water (2% wt) (16 v) and MeOH (10% wt) at 23° C. The resulting suspension was warmed to 40° C. and stirred for 24 h. The mixture was cooled to 23° C., stirred and the suspension was filtered under vacuum (Por. 4).

Obtained material: Columnar particles from ˜1 μm up to 20 μm in length. Most of the particles are smaller than 5 μm. No agglomerates. XRPD: Form A. Filtration time on lab scale: >10 min

### Example 4: Crystallization of Compound 1 with TPGS-1000 and MeOH

500 mg of compound 1 was added to MeOH (16 v). A clear solution was obtained at 60° C. To this solution was added a solution of TPGS-1000 in water (5% wt) (8 v). The reaction mixture was cooled to 23° C., stirred and the resulting suspension was filtered under vacuum (Por. 4).

Obtained material: Aggregates/agglomerates (up to 50 μm) of columnar particles. XRPD: Form A. Filtration time on lab scale: 20 s

### Example 5: Crystallization of Compound 1 with PEG200 and MeOH

500 mg of compound 1 was added to MeOH (16 v). A clear solution was obtained at 60° C. To this solution was added a solution of PEG200 in water (5% wt) (8 v). The reaction mixture was cooled to 23° C., stirred and the resulting suspension was filtered under vacuum (Por. 4).

Obtained material: Aggregates (up to 750 μm) in diameter of platy, particles up to 200 μm. XRPD: Form B. Filtration time on lab scale: 5 s

### Example 6: Crystallization of Compound 1 with TPGS-750-M and MeOH

500 mg of compound 1 was added to MeOH (16v). A clear solution was obtained at 60° C. To this solution was added a solution of TPGS-750-M in water (2% wt) (8 v). The reaction mixture was cooled to 23° C., stirred and the resulting suspension was filtered under vacuum (Por. 4).

Obtained material: Small needles. Most of the particles are in a range of 10-20 μm. XRPD: Form A. Filtration time on lab scale: 80 s

### Example 7: Crystallization of Compound 1 with Solutol-HS and MeOH

500 mg of compound 1 was added to MeOH (16 v). A clear solution was obtained at 60° C. To this solution was added a solution of Solutol-HS in water (2% wt) (8 v). The reaction mixture was cooled to 23° C., stirred and the resulting suspension was filtered under vacuum (Por. 4).

Obtained material: Thin plates and aggregates, small needles. Large distribution of particule shape. Most of the particles are in a range of 20-50 μm. XRPD: Form A. Filtration time on lab scale: 70 s

### Example 8: Crystallization of Compound 1 with Tween 80 and MeOH

500 mg of compound 1 was added to MeOH (16 v). A clear solution was obtained at 60° C. To this solution was added a solution of Tween 80 in water (2% wt) (8 v). The reaction mixture was cooled to 23° C., stirred and the resulting suspension was filtered under vacuum (Por. 4).

Obtained material: Thin plates and aggregates, small needles. Most of the particles are in a range of 20-40 μm. XRPD: Form A. Filtration time on lab scale: 60 s

### Example 9: Crystallization of Compound 1 with Triton X100 and MeOH

500 mg of compound 1 was added to MeOH (16 v). A clear solution was obtained at 60° C. To this solution was added a solution of Triton X100 in water (2% wt) (8 v). The reaction mixture was cooled to 23° C., stirred and the resulting suspension was filtered under vacuum (Por. 4).

Obtained material: Aggregates, small plates. Big aggregates up to 100 μm. Most of the particles are in a range of 10-20 μm. XRPD: Form A. Filtration time on lab scale: 7 s

### Example 10: Crystallization of Compound 1 with TPGS-1000 and MeOH

500 mg of compound 1 was added to MeOH (16v). A clear solution was obtained at 60° C. To this solution was added a solution of TPGS-1000 in water (2% wt) (8 v). The reaction mixture was cooled to 23° C., stirred and the resulting suspension was filtered under vacuum (Por. 4).

Obtained material: Small, dispersed particles. Most of the particles are in a range of 1-5 μm. XRPD: Form A. Filtration time on lab scale: 45 s

### Example 11: Crystallization of Compound 1 with PEG200 and MeOH

500 mg of compound 1 was added to MeOH (16 v). A clear solution was obtained at 60° C. To this solution was added a solution of PEG200 in water (5% wt) (8 v). The reaction mixture was cooled to 23° C., stirred and the resulting suspension was filtered under vacuum (Por. 4).

Obtained material: Plates up to 200 μm. Most of the particles are in a range of 50-100 μm. XRPD: Form B. Filtration time on lab scale: 5 s

### Example 12: Crystallization of Compound 1 with PEG600 and MeOH

500 mg of compound 1 was added to MeOH (16 v). A clear solution was obtained at 60° C. To this solution was added a solution of PEG600 in water (5% wt) (8 v). The reaction mixture was cooled to 23° C., stirred and the resulting suspension was filtered under vacuum (Por. 4).

Obtained material: Mix of Aggregated needles and plates. Most of the particles are in a range of 10-20 μm. XRPD: Form A. Filtration time on lab scale: 15 s

### Example 13: Crystallization of Compound 1 with TPGS-750-M

500 mg of compound 1 was added to solution of TPGS-750-M in water (5% wt) (16 v) at 23° C. The resulting suspension was warmed to 40° C. and stirred for 24 h. The mixture was cooled to 23° C., stirred and the suspension was filtered under vacuum (Por. 4).

Obtained material: Mix of broken plates and fine needles. Most of the particles are in a range of 20-40 μm. Very fine needles can be observed (<5 μm). XRPD: Form A. Filtration time on lab scale: >5 min

1. A method of producing crystals or aggregates of organic compounds, comprising providing a composition comprising said organic compound and a surfactant-water mixture, and inducing crystallization or aggregation of the organic compound.

2. The method according to claim 1, wherein crystallization or aggregation of the organic compound is induced by reducing its solubility.

3. The method according to claim 1, wherein crystallization or aggregation of the organic compound is induced by reducing the temperature of the composition.

4. The method according to claim 1, wherein the surfactant is a non-ionic surfactant.

5. The method according to claim 1, wherein the surfactant comprises a hydrophilic part and a hydrophobic part, and wherein the hydrophilic part of the surfactant comprises a polyethylene glycol moiety.

6. The method according to claim 1, wherein the surfactant is selected from the group consisting of (DL-α-) tocopherol polyethylene glycol succinate (TPGS) such as TPGS-750-M, TPGS-1000 and TPGS-1500; Triton X-100, polyethylene glycol alkyl ether such as Brij, polyethylene glycol ester such as polyethylene glycol (15)-hydroxystearate (Solutol HS 15), Tween such as Tween 20 or Tween 80, sodium dodecyl sulfate (SDS), cetyltrimethylammonium bromide (CTAB), phase transfer surfactants (PTS) (e.g. sodium deoxycholate), polyoxyethanyl ubiquinol sebacate (PQS) and functionalized PQS, polyethylene glycol (PEG) such as PEG200 and PEG600, and various derivatives such as C4-azo-PEG, octanoic acid and other long alkyl chain acids, and b-sitosterol methoxyethyleneglycol succinate (Nok).

7. The method according to claim 1, wherein the concentration of the surfactant in the surfactant-water mixture is above its critical micellar concentration.

8. The method according to claim 1, wherein the concentration of the surfactant in the surfactant-water mixture is 0.5 to 5% (w/w).

9. The method according to claim 1, wherein the organic compound is a small molecule having a molecular weight of 2,000 Dalton or less.

10. The method according to claim 1, wherein the organic compound is an intermediate or final product of a chemical synthesis process.

11. The method according to claim 1, wherein the step of providing a composition comprising said organic compound and a surfactant-water mixture comprises solving the organic compound in the surfactant-water mixture.

12. The method according to claim 1, wherein the composition further comprises an organic solvent.

13. The method according to claim 12, wherein the organic solvent is water-miscible.

14. The method according to claim 12, wherein the organic solvent is an aprotic organic solvent.

15. The method according to claim 12, wherein the organic solvent is selected from the group consisting of alcohol such as C1-6 alcohol, in particular methanol and ethanol; acetone; tetrahydrofuran (THF); methyl tetrahydrofuran; and pyridine.

16. The method according to claim 12, wherein the step of providing a composition comprising said organic compound and a surfactant-water mixture comprises solving the organic compound in an organic solvent and then adding the surfactant-water mixture.

17. The method according to claim 1, further comprising the step of obtaining the crystallized or aggregated organic compound from the composition.

18. The method according to claim 17, wherein the crystallized or aggregated organic compound is obtained by filtration.

19. The method according to claim 1, wherein the composition is of industrial scale.

20. The method according to claim 19, wherein the volume of the composition is at least 1 l, preferably at least 10 l.

21. A method for changing the morphology of crystals or aggregates of an organic compound, comprising solving the crystals or aggregates of the organic compound in a composition comprising a surfactant-water mixture, and inducing crystallization or aggregation of the organic compound.

22. The method according to claim 21, wherein the method for producing crystals or aggregates according to claim 1 is used for crystallization or aggregation of the organic compound.

23. A method for synthesizing an organic compound, comprising chemically modifying a starting compound to produce said organic compound, and isolating and/or purifying the obtained organic compound using the method for producing crystals or aggregates according to claim 1.

24. The method according to claim 23, wherein the organic compound is an intermediate product or a final product.
